(19) United States
(12) Patent Application Publication
(10) Pub. No.: US 2002/0094565 A1
(43) Pub. Date: Jul. 18, 2002

(54) Method for purifying nucleic acid and apparatus using the same

(75) Inventors: Toshinari Sakurai, Hitachinaka-shi (JP); Kenji Yasuda, Tokyo (JP); Koichi Matsumoto, Hitachinaka-shi (JP)

Correspondence Address: MATTINGLY, STANGER & MALUR, P.C., Suite 370, 1800 Diagonal Road, Alexandria, VA, 22314, US

(73) Assignee: Hitachi, Ltd.

(21) Appl. No.: 10/029,981

(22) Filed: Dec. 31, 2001

Related U.S. Application Data
(62) Division of application No. 09/268,637, filed on Mar. 16, 1999.

(30) Foreign Application Priority Data
Mar. 19, 1998 (JP) 10-70201

(51) Int. Cl. C12M 3/00; A61K 31/70; A01N 43/04; C12M 1/00

(52) U.S. Cl. 435/285.3; 514/44; 435/283.1

(57) ABSTRACT

A sample and a binding enhancer are supplied to a treating container using a pipette tip connected to a connecting nozzle. A mixture in the treating container is sucked into a nucleic acid trapping pipette tip, and nucleic acid in the mixture is trapped by a solid phase substance contained in the pipette tip. After the solid phase substance is washed with a washing solution, an eluting solution is sucked into the nucleic acid trapping pipette tip, and a liquid containing the eluted nucleic acid is discharged to a purified product container.

### BACKGROUND OF THE INVENTION

1. Field of the Invention

The present invention relates to a method of purifying a nucleic acid and an apparatus for purifying a nucleic acid, and particularly to a method and an apparatus suitable for isolating and extracting a nucleic acid contained in a biological sample from coexisting substances.

2. Description of the Related Art

Various kinds of technologies concerning genes have been developed by progress of the molecular biology, and various kinds of disease genes have been isolated and identified by the technologies. As a result, methods of the molecular biological technology have been employed for diagnoses or inspections in the medical field to make diagnoses not practicable in the past diagnosable and to substantially shorten inspection period.

The progress described above largely depends on a gene amplification method, and particularly on a polymerase chain reaction method (hereinafter referred to as a PCR method). Since the PCR method can sequence-specifically amplify a nucleic acid in a solution, existence of a very small amount of viruses, for example, in blood serum can be indirectly proved by amplifying and detecting the nucleic acid of genes of the viruses. However, there are some problems when the PCR method is used for an ordinary inspection in the clinical field. An important thing among them is an extraction and a purification processes in the preparation treatment in order to maintain accuracy. In regard to the purification of a nucleic acid, some methods are proposed.

Japanese Patent Application Laid-Open No.2-289596, which corresponds to U.S. Pat. No. 5,234,809, proposes that silica beads capable of binding with a nucleic acid under existing of a chaotropic agent is used as a nucleic acid binding solid phase substance. In Japanese Patent Application Laid-Open No.2-289596 described above, the purifying method comprises the steps of adding a sample containing a nucleic acid into a reaction container containing a suspension of silica beads and a guanidine thiocyanate buffer as a chaotropic agent and mixing them; centrifuging a complex of the nucleic acid binding with the silica beads; then disposing the supernatant; adding a wash solution to the remaining complex and washing the remaining complex using a vortex mixer; washing the washing the remaining complex with an ethanol solution; then washing the washing the remaining complex with acetone; drying the complex by removing the acetone; and eluting the nucleic acid by adding an eluting buffer to the dried complex to collect the nucleic acid.

Japanese Patent Application Laid-Open No.8-320274 proposes a method of isolating a DNA using a plurality of containers and a plurality of pipette tips for a single sample. The following is described in Japanese Patent Application Laid-Open No.8-320274. That is, a first tip is attached to a connecting pipette nozzle moved by a moving mechanism, and a sample is sucked in the first tip. Then, a filter for removing shells of hemacytes is fit to the bottom end of the first tip, and the sample in the first tip is discharged into a first container through the filter. After that, the filter and the first tip are detached from the connecting pipette nozzle, and a second tip is attached to the bottom end of the connecting pipette nozzle, and the sample in the first container is sucked into the second tip. Then a silica membrane filter for trapping DNA is fit to the bottom end of the second tip, and the sample in the second tip is discharged to a second container through the silica membrane filter to trap the DNA by the silica membrane filter and to discharge impurities. After that, the connecting pipette nozzle is moved to a third container containing a washing solution, and the silica membrane filter trapping the DNA is detached from the second tip and immersed into the washing solution in the third container. Then, a third tip is attached to the connecting pipette nozzle which the second tip has been detached from, and the silica membrane filter in the third container is attached to the bottom end of the third tip, and the mixture of the washing solution and the DNA is sucked into the third tip, and after that the mixture is discharged into a fourth container.

Further, Japanese Patent Application Laid-Open No.7-250681 proposes a method of purifying a DNA extract solution using a cartridge container having a filter member of a quadruple structure in which a glass powder layer is interposed between two glass fiber filters and two membrane filters. In Japanese Patent Application Laid-Open No.7-250681, a prepared culture culture medium containing DNA is pre-treated to gather a transformant in the trap filter, and a lysis agent is added to elute plasmid DNA out of the cells. Thus, the obtained plasmid DNA is used as a sample to be purified. In the purifying process, a purifying agent and a chaotropic iongenic agent (sodium iodide) are added to the cartridge container, and the cartridge container is treated with vacuum or centrifuging treatment to adsorb the plasmid DNA to the glass powder layer of the cartridge container. Then, a wash buffer is added to the cartridge container to be washed through vacuum or centrifuging treatment. After that, an elution buffer is added to the cartridge container, and the cartridge container is treated with vacuum or centrifuging treatment to elute only the plasmid DNA.

Among the prior arts described above, the method of Japanese Patent Application Laid-Open No.2-289596 is difficult to automate the purifying process because the centrifuging operation is necessary. Further, the method of Japanese Patent Application Laid-Open No.7-250681 is difficult to be automated because the vacuum or the centrifuging operation must be performed several times. Furthermore, the method of Japanese Patent Application Laid-Open No.8-320274 is low in the trapping ratio due to short contact time between the sample and the silica membrane because the apparatus is designed so that DNA is trapped when the sample is discharged from the second tip through the silica membrane.

### SUMMARY OF THE INVENTION

An object of the present invention is to provide a method of purifying nucleic acid and an apparatus for purifying nucleic acid which can sufficiently maintain contact time between a sample and a solid phase substance at trapping of the nucleic acid though the apparatus can be easily automated.

Further, another object of the present invention is to provide a method of purifying nucleic acid and an apparatus for purifying nucleic acid in which processes of trapping nucleic acid to a solid phase substance, washing the trapped substance and eluting the trapped substance can be performed using a single solid phase substance containing tip for sucking and discharging a liquid.

A method of purifying nucleic acid in accordance with the present invention comprises the steps of detachably connecting a nucleic acid trapping pipette tip containing a solid phase substance containing silica to a liquid sucking-and-discharging movable nozzle; sucking a mixture of a substance for enhancing binding of the nucleic acid to the solid phase substance into the nucleic acid trapping pipette tip connected to the liquid sucking-and-discharging movable nozzle from a predetermined container; discharging the liquid in the nucleic acid trapping pipette tip after binding the nucleic acid in the sucked mixture to the solid phase substance; washing the solid phase substance in the state of binding the nucleic acid and the inside of the nucleic acid trapping pipette tip by sucking a washing solution into the nucleic acid trapping pipette tip after discharging the liquid and then by discharging said washing solution from the nucleic acid trapping pipette tip; sucking an eluting solution into the nucleic acid trapping pipette tip after washing; and discharging the eluting solution containing the nucleic acid removed from the solid phase substance into a purified product container.

In a preferable embodiment in accordance with the present invention, the nucleic acid trapping pipette tip connected to the liquid sucking-and-discharging movable nozzle is exchanged to a new nucleic acid trapping pipette tip every change of a sample to be treated. Further, in the process of sucking the sample mixture in the nucleic acid trapping pipette tip, the number of times that the mixture is brought in contact with the solid phase substance is increased by repeating the process that the mixture once sucked is discharged to the original predetermined container and then sucked the mixture into the nucleic acid trapping pipette tip again. During the washing process, by dividing the amount of the washing solution into several parts, operation of sucking and discharging the washing solution is repeated plural cycles. Similarly, during the eluting process, by dividing the amount of the eluting solution into several parts, operation of sucking and discharging the eluting solution is repeated plural cycles.

An apparatus for purifying nucleic acid in accordance with the present invention comprises a nucleic acid trapping pipette tip containing a solid phase substance containing silica capable of contacting with a liquid; a liquid sucking-and-discharging movable nozzle detachably connecting the nucleic acid trapping pipette tip; a treating container capable of containing a mixture of a substance for enhancing binding of the nucleic acid with the solid phase substance and a sample containing the nucleic acid; a means for supplying a washing solution to the treating container; a means for supplying a eluting solution to the treating container; a purified product container for receiving a purified product of the nucleic acid; a transferring means for connecting the nucleic acid trapping pipette tip in an unused state to the liquid sucking-and-discharging movable nozzle and for moving the nucleic acid trapping pipette tip in a connected state to positions of the treating container and the purified product container; a liquid sucking-and-discharging operating means for sucking and discharging said mixture from and to the nucleic acid trapping pipette tip connected to the liquid sucking-and-discharging movable nozzle, and sucking and discharging the washing solution, and then sucking and discharging the eluting solution; and a pipette tip detaching means for detaching the nucleic acid trapping pipette tip from the liquid sucking-and-discharging movable nozzle after discharging the eluting solution from the nucleic acid trapping pipette tip to the purified product container.

### BRIEF DESCRIPTION OF DRAWINGS

FIG. 1 is a plan view showing an embodiment of an apparatus for purifying nucleic acid in accordance with the present invention.

FIG. 2 is a schematic external view showing the apparatus of FIG. 1.

FIG. 3 is a block diagram explaining the structure of an electric system in the apparatus of FIG. 1.

FIG. 4 is a schematic view showing the structure of a pipetter in the apparatus of FIG. 1.

FIG. 5 is a view explaining the operation of attaching a pipette tip to a connecting nozzle in the apparatus of FIG. 1.

FIG. 6 is a view explaining the operation of detaching the pipette tip from the connecting nozzle.

FIG. 7 is a schematic view showing an embodiment of a nucleic acid trapping pipette tip used in the apparatus of FIG. 1.

FIG. 8 is a view showing another embodiment of a nucleic acid trapping pipette tip.

FIG. 9 is a view showing a still other embodiment of a nucleic acid trapping pipette tip.

### DETAILED DESCRIPTION OF THE PREFERRED EMBODIMENTS

As samples containing nucleic acid to be purified, there are body samples such as whole blood, blood serum, phlegm and urine; biological samples such as cultured cells and cultured bacterium; and nucleic acid held in a gel after electrophoresis; reaction products such as DNA amplifying enzyme; substance containing nucleic acid in a coarse state and so on. The nucleic acid here includes deoxyribonucleic acid (DNA) and ribonucleic acid (RNA) having a double-stranded, a single-stranded structure, or having a partly double-stranded or a partly single-stranded structure.

As the substance enhancing binding of nucleic acid to the solid phase substance containing silica, it is preferable that the substance has a small absorbance near 260 nm wavelength which is the absorbance peak of nucleic acid. Because an absorbance at 260 nm wavelength is generally measured in the assaying of purity or quantity of nucleic acid using a spectrophotometer. In addition, a substance containing chiocyanic acid is not preferable from the viewpoint of handling because it produces a lethal gas when it reacts with an acid. In the preferable embodiment of the present invention, guanidine hydrochloride (GuHCl) is used as a chaotropic agent with taking these points into consideration. In a case of using guanidine hydrochloride, it is preferable that the final concentration is 4 to 6 mol/l.

As the solid phase substance containing silica contained in the nucleic acid trapping pipette tip, any substance containing silicon oxide such as glass beads, silica beads, a quartz filter, quartz wool, or crushed things of these material, diatomaceous earth or the like may be used. However, in order to prevent the solid phase substance from releasing from the pipette tip, an inner diameter of a top end portion of the pipette tip is made small and an outer diameter of the solid phase substance beads is made larger than the inner diameter of the top end portion of the pipette tip. Otherwise, a holding member with holes having a diameter smaller than the outer diameter of the solid phase substance beads is placed at a position near the top end portion of the pipette tip. By doing so, the solid phase substance can be in contact with the sample with a large contact area when the sample is sucked into the pipette tip, and at the same tome the solid phase substance can be held in the pipette tip.

Eluting of the nucleic acid from the solid phase substance can be performed by mixing a low concentration saline or water to the solid phase substance after the washing process. Since the nucleic acid is mass-transferred from the solid phase to the water phase by this operation, a purified nucleic acid aqueous solution can be obtained by collecting the water phase substance to a purified product container.

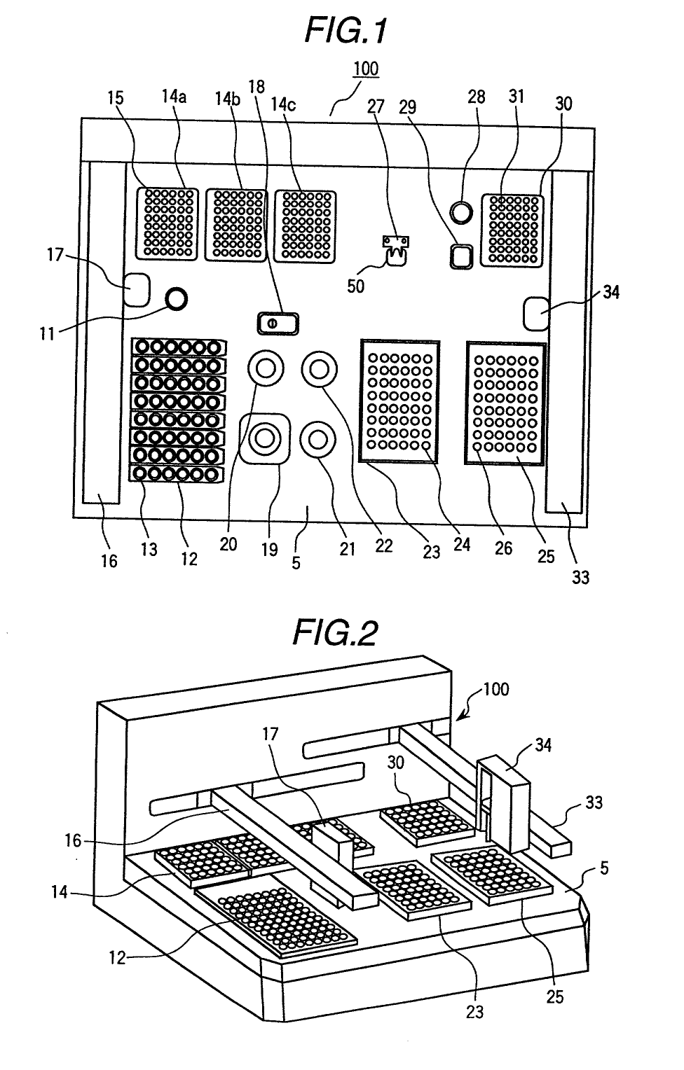

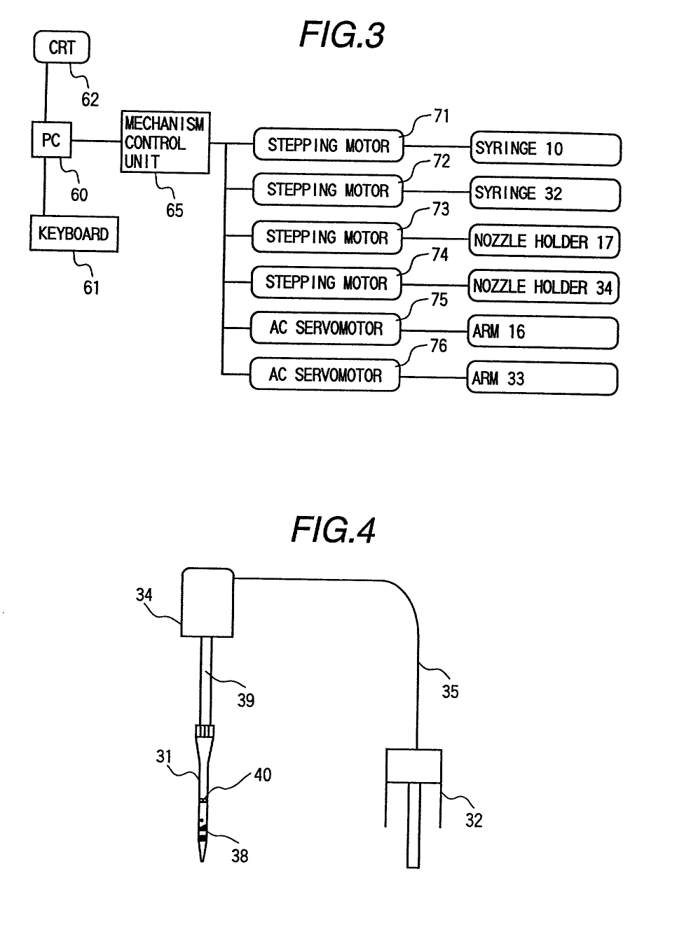

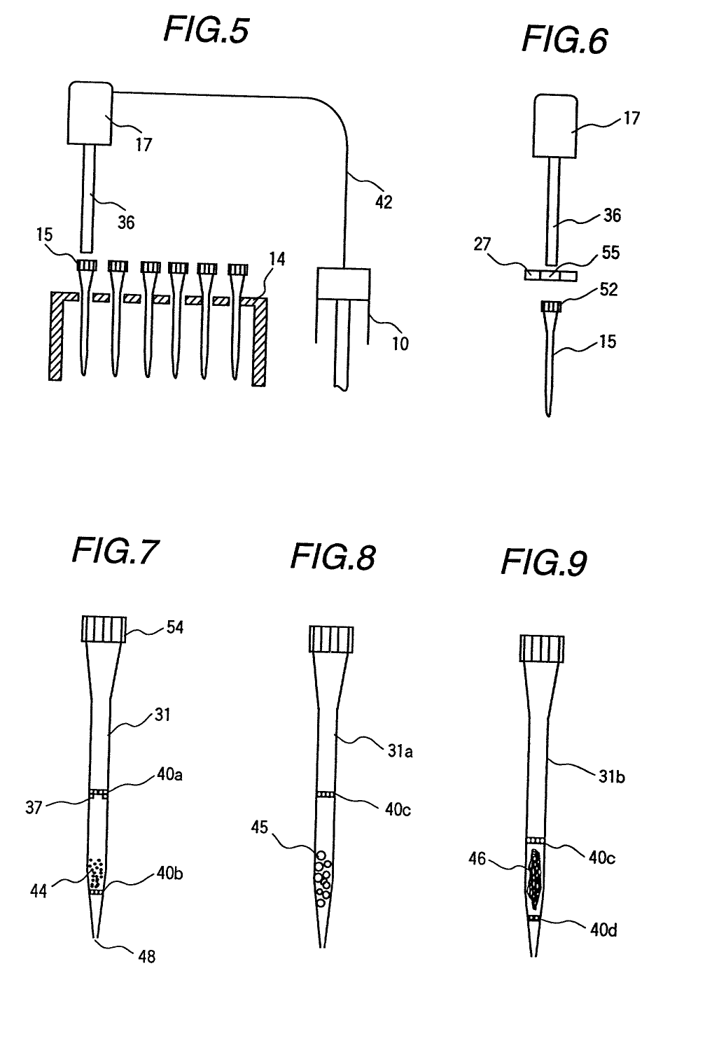

An embodiment of a nucleic acid purifying apparatus in accordance with the present invention will be described below, referring to FIG. 1 to FIG. 7. FIG. 1 is a plan view showing an embodiment of the apparatus. FIG. 2 is an external view of the apparatus. FIG. 3 is a block diagram of the electric system. FIG. 4 is a schematic view showing the structure of a pipetter having a nucleic acid trapping pipette tip connected to the pipetter. FIG. 5 is a view explaining the operation of attaching the liquid pipette tip to a connecting nozzle. FIG. 6 is a view explaining the operation of detaching the pipette tip from the connecting nozzle. FIG. 7 is a view showing the construction of the nucleic acid trapping pipette tip.

Referring to FIG. 1 and FIG. 2, the nucleic acid purifying apparatus 100 comprises two arms 16, 33 movable in a horizontal direction (X-direction). A nozzle holder 17 for holding a connecting pipette nozzle 36 (FIG. 5) is attached in one of the arms 16 movably in a horizontal direction (Y-direction) along the lateral direction of the arm 16. A nozzle holder 34 for holding a liquid sucking-and-discharging pipette nozzle 39 (FIG. 4) is attached in the other of the arms 33 movably in a horizontal direction (Y-direction) along the lateral direction of the arm 33. Both of the nozzle holders 17, 34 can be operated in the vertical direction (Z-direction) to the corresponding arms 16, 33, respectively. Since a horizontally movable zone of the arm 16 and a horizontally movable zone of the arm 33 are partly overlapped with each other, Installation levels of the arms are different from each other.

Three tip racks 14a, 14b, 14c mounting many unused pipette chips 15 are set to predetermined areas on a work surface 5 of the main frame. As shown in FIG. 5, each of these tip racks 14 has through holes capable of letting the pipette tip 15 insert to the hole, and is of a box-shape having a height high enough to prevent the top end of the pipette tip from touching the work surface 5 or the bottom surface of the pipette tip rack. Each of the pipette tip racks 14a, 14b, 14c can hold forty-eight of the pipette tips 15 at maximum.

Further, a pipette tip rack 30 mounting many unused nucleic acid trapping pipette chips 31 is set to a predetermined area on the work surface 5. The shape of the pipette tip rack 30 is the same as that of the pipette tip rack 14 described above. In this embodiment, the pipette tip rack 30 can hold forty-eight of the nucleic acid trapping pipette tips 31 at maximum.

Sample racks 12 respectively holding several sample containers 13 containing a sample body to be treated, that is, a sample containing nucleic acid are set to predetermined areas on the work surface 5. In this embodiment, each of the sample racks 12 can hold six of the sample containers 13. Eight or more of the sample racks 12 can be set.

Further, a container rack 23 holding many unused treating containers 24 is set to a predetermined area on the work surface 5. The container rack 23 can hold forty-eight of the treating containers 24 at maximum. Furthermore, a container rack 23 holding many unused purified product containers 26 is set to a predetermined area on the work surface 5. This purified product container is for collecting a purified solution containing nucleic acid for each sample. In this embodiment, the container rack 25 can hold forty-eight of the purified product containers 26 at maximum.

On the work surface 5, a solution receiving portion 11 which receives water discharged from the connecting pipette nozzle 36 at priming, and becomes a home position of the connecting pipette nozzle 36; a washing portion 18 for washing the pipette tip 15 for pipetting work; a pipette tip detaching implement 27 for detaching the pipette tip 15 connected to the connecting pipette nozzle 36 and the nucleic acid trapping pipette tip 31 connecting to the liquid sucking-and-discharging movable nozzle 39 from the corresponding nozzles; a solution receiving portion 28 which receives water discharged from the liquid sucking-and-discharging movable nozzle 39 at priming, and becomes a home position of the movable nozzle 39; and a waste liquid port 29 for discharging unnecessary solution from the nucleic acid trapping pipette tip 31 are provided.

Further, in each of predetermined positions on the work surface 5, a washing solution bottle 19 containing a washing solution for washing the solid phase substance in the nucleic acid trapping pipette tip 31; an eluting solution bottle 20 containing an eluting solution for eluting nucleic acid bound to the solid phase substance; a diluting solution bottle 21 containing a diluting solution; and a binding enhancer bottle 22 containing a solution of a binding enhancing agent for enhancing binding nucleic acid to the solid phase substance are set.

A syringe pump 10 shown in FIG. 5 and a syringe pump 32 shown in FIG. 4 are respectively installed on the main bases, and control of sucking operation and delivering operation of each of the pumps is independently performed. As shown in FIG. 5, the connecting pipette nozzle 36 held to the nozzle holder 17 is communicated to the liquid sucking-and-discharging syringe pump 10 through a flexible pipe 42. The inside of the connecting pipette nozzle 36 and the inside of the pipe 42 are filled with purified water, and the syringe pump 10 is connected to a purified water supply source, not shown. As shown in FIG. 4, the movable nozzle 39 held to the nozzle holder 34 is connected to the syringe pump 32 through a flexible pipe 35. The inside of the movable nozzle 39 and the inside of the pipe 35 are filled with purified water, and the syringe pump 32 is connected to a purified water supply source, not shown.

Attaching to connect the pipette tip 15 to the connecting pipette nozzle 36 or attaching to connect the nucleic acid trapping pipette tip 31 to the movable nozzle 39 is performed on the corresponding tip rack 14 or 30 by moving the nozzle downward to fit the pipette tip to top end of the nozzle. When the pipette tip 15 connected to the connecting pipette nozzle 36 or the nucleic acid trapping pipette tip 31 connected to the movable nozzle 39 is detached from the corresponding nozzle, the pipette tip detaching implement 27 is used. As shown in FIG. 1 and FIG. 6, the pipette tip detaching implement 27 has a plate-shaped member at a position in a predetermined level, and in this plate-shaped member there is formed a slit 55 width of which is smaller than the outer diameter of a head portion 52 of the pipette tip 15 and larger than the outer diameter of the connecting pipette nozzle 36 and the outer diameter of the movable nozzle 39. The nozzle 36 or 39 is horizontally moved with keeping the head portion 52 or 54 of the pipette tip at a positional level lower than the positional level of the slit 55 to be inserted into the slit, and then the nozzle holder 17 or 34 is moved upward to bring the head portion 52 or 54 in contact with the lower surface of the plate-shaped member. As the nozzle holder is moved further upward, the pipette tip 15 or 31 is detached from the nozzle to fall down. The detached tip falls into the pipette tip waste port 50 (FIG. 1) and is collected in a collecting box, not shown.

FIG. 3 shows the structure of the electric system in the nucleic acid purifying apparatus of FIG. 1. A keyboard 61 as an operating panel for inputting operating conditions and sample information; a CRT 62 as a display unit for displaying input information, alarm information and so on; and a mechanism control unit 65 for controlling mechanism portions of the apparatus are connected to a personal computer (PC) 60 as an operating control unit. The mechanism control unit 65 controls a piston drive stepping motor 71 for performing sucking-and-delivering operation of the syringe pump 10; a piston drive stepping motor 72 for performing sucking-and-delivering operation of the syringe pump 32; a stepping motor 73 for horizontally and vertically moving the nozzle holder 17; a stepping motor 74 for horizontally and vertically moving the nozzle holder 34; an AC servo motor 75 for horizontally moving the arm 16; an AC servo motor 76 for horizontally moving the arm 33. Each portion of the purifying apparatus is operated according to a predetermined program.

FIG. 7 shows an example of the nucleic acid trapping pipette tip 31. The nucleic acid trapping pipette tip 31 is formed in such that the head portion 54 has an inner diameter capable of air-tightly fit to the top end of the movable nozzle 39, and that the inner diameter of the lower portion is gradually thinned toward the top end 48. The pipette tip 31 is made of a transparent or translucent synthetic resin. A disk-shaped preventive member 40b for preventing the solid phase substance from flow out is inserted through press-fitting in the side of the top end of the pipette tip 31, and a disk-shaped preventive member 40a is arranged in the side of the head portion 54. Each of the preventive members 40a, 40b has many holes capable of allowing liquid and gas easily pass through, but capable of preventing the solid phase substance from flowing out. As the material, the preventive members 40a, 40b employs poly(vinylidene fluoride) which is small in nonsingular adsorption and has a hydrophilic property. Since poly(vinylidene fluoride) can make the nonsingular adsorption of protein or nucleic acid small, the material less affects the purifying degree or the yield of nucleic acid. The preventive member 40a has a plurality of projected insertion assisting guides 37 for making insertion to the pipette tip 31 easy in the lower surface side of the member. Powder 44 of flint glass (a product of Wakou Pure Chemicals Co.) is filled in a space surrounded by the preventive members 40a and 40b as the solid phase substance. The flint glass has a high content of silica.

Purifying operation of nucleic acid using the embodiment of FIG. 1 will be described below.

In prior to starting the purifying operation of a sample containing nucleic acid, a solution was prepared by diluting a commercial purified product of pBR322DNA (a product of Fermentas Co.) to a predetermined concentration with a Tris-EDTA buffer (pH 7.5, TE buffer), and was contained in a sample container 13. The sample container 13 was held by a sample rack, and the sample rack was set in the sample area on the apparatus 100 of FIG. 1. The pipette tip rack 14 having the pipette tips 15, the pipette tip rack 30 having the nucleic acid trapping pipette tips 31, the bottles 19, 20, 21 and 22, the container rack 23 having the treating containers 24, and the container rack 25 having the purified product containers 26 were respectively set to the corresponding positions, and then the operation using the purifying apparatus was started.

Initially, by operating the nozzle holder 17, the connecting pipette nozzle 36 positioned at the liquid receiving portion 11 is moved above the sample tip rack 14a, and a first pipette tip is fit to the connecting pipette nozzle 36. Then, the attached pipette tip 15 is moved above the binding enhancer bottle 22 and moved down into the bottle, and a predetermined amount of guanidine hydrochloride solution is sucked into the pipette tip 15 by sucking operation of the syringe pump 10. The pipette tip 15 is moved up from the binding enhancer bottle 22, and a small amount od air is sucked into the top end of the pipette tip. The pipette tip 15 is moved to the washing portion 18, and the outer wall of the pipette tip 15 is washed by spraying a washing solution onto the outer wall of the pipette tip. Next, the connecting pipette nozzle 36 is moved to a first sample container 13 on the sample rack 12, and the pipette tip 15 is moved down in the sample container to suck a predetermined amount of the sample into the pipette tip 15 by sucking operation of the syringe pump 10. By doing so, layers of the guanidine hydrochloride solution, the air and the nucleic acid containing solution are formed in the pipette tip 15.

The pipette tip 15 containing the sucked sample is moved above a first treating container 24 on the container rack 23, and the total amount of the sample and the guanidine hydrochloride solution in the pipette tip 15 is discharged into the treating container 24. After the discharging, the total amount of the discharged solution is sucked into the same pipette tip 15 and the sucked solution is discharged into the first treating container 24. This process is repeated one or more cycles. By doing so, the nucleic acid containing solution and the binding enhancer are mixed. Then, the connecting pipette nozzle 36 is moved to the pipette tip detaching implement 27 to remove the used pipette tip 15 from the connecting pipette nozzle 36 according to the detaching operation described previously. Next, the connecting pipette nozzle 36 is returned to the position of the liquid receiving portion 11, and a predetermined amount of purified water is discharged from the connecting pipette nozzle 36 and then a small amount of air is sucked in the top end of the connecting pipette nozzle 36. Thus, the connecting pipette nozzle stands by for the next operation command to the nozzle holder 17.

While the connecting pipette nozzle 36 is performing the mixing operation, the nucleic acid trapping movable nozzle 39 is moved from the liquid receiving portion 28 to a position of a first nucleic acid trapping pipette tip 31 on the pipette tip rack 30 by operation of the arm 33 and the nozzle holder 34, and the nucleic acid trapping pipette tip 31 is fit to the top end of the movable nozzle 39. After that, the movable nozzle 39 in the state of having the nucleic acid trapping pipette tip 31 fit is moved to the position of the first treating container 24 on the container rack 23, and the nucleic acid trapping pipette tip 31 is moved down to the first treating container 24 to suck the total amount of the mixture of the sample and the binding enhancer contained in the first treating container into the nucleic acid trapping pipette tip 31 by sucking operation of the syringe pump 32. By doing so, the mixture is brought in contact with the surface of the glass powder 44 as the solid phase substance in the pipette tip 31. After that, the sucked mixture is discharged into the first treating container 24, and the discharged mixture is sucked into the same nucleic acid trapping pipette tip 31 again. By repeating the operation of sucking and discharging the mixture plural cycles, the number of cycles of contacting between the solid phase substance surface and the mixture is increased and accordingly the adsorption efficiency of the nucleic acid by the solid phase substance is increased.

After repeating sucking and discharging predetermined cycles, the total amount of the mixture is finally sucked into the first nucleic acid trapping pipette tip 31, and the pipette tip 31 is moved to the waste liquid port 29 to discharge the remaining liquid after nucleic acid adsorption into the waste liquid port 20 by discharging operation of the syringe pump 32. The movable nozzle 39 in the state of having the nucleic acid trapping pipette tip 31 connected is moved to the position of the liquid receiving portion 28 to stand by for the next operating command to the nozzle holder 34.

While the nucleic acid trapping pipette tip 31 is operating sucking and discharging of the mixture, the next operation command is output to the nozzle holder 17. That is, the connecting pipette nozzle 36 is moved from the position of the liquid receiving portion 11 to the position of the first pipette tip on the pipette tip rack 14b, and the connecting pipette nozzle 36 is moved down to fit the first pipette tip to the top end. By the time when the nucleic acid trapping pipette tip 31 is moved to the waste port 29, the connecting pipette nozzle 36 having the pipette tip 15 connected is moved to the position of the washing solution bottle 19 to suck the washing solution by an amount capable of using the total amount in plural washing operations into the pipette tip. Next, the connecting pipette nozzle 36 is moved above the first treating container 24 emptied by sucking the mixture on the container rack 23, and the part (the amount for one washing operation) of the washing solution is discharged from the pipette tip 15 into the first treating container 24 by the delivering operation of the syringe pump 10. After discharging the washing solution, the pipette tip 15 is moved to the liquid receiving portion 11 to stand by.

Successively, the nucleic acid trapping pipette tip 31 standing by on the liquid receiving portion 28 is moved to the first treating container containing the washing solution, and the washing solution of the treating container 24 is sucked into the nucleic acid trapping pipette tip 31 by sucking operation of the syringe pump 32. The washing solution once sucked is discharged to the first treating container 24, and then sucked into the pipette tip 31 again. After repeating the operation of discharging and sucking twice, the movable nozzle 39 in a sate of finally sucking the washing solution is moved to the waste liquid port 29, and the used washing solution is discharged in the waste port 29. By such washing operation, the inner wall of the nucleic acid trapping pipette tip 31 and the surface of the solid phase substance are washed. Then, the pipette tip 15 standing by at the liquid receiving portion 11 is moved to the first treating container 24, and a part or the total of the washing solution held in the pipette tip 15 is discharged into the first treating container. The pipette tip 15 after discharging the washing solution is moved to the position of the pipette tip detaching implement 27, and detached from the connecting pipette nozzle 36. The washing solution newly added to the first treating container 24 is sucked into the nucleic acid trapping pipette tip 31 to used for performing the second washing operation. The second washing operation is performed in the same steps as in the first washing operation. The third washing operation may be performed, if necessary. The nucleic acid trapping pipette tip 31 after completing of the washing operation by discharging the washing solution to the waste liquid port 29 is moved to the liquid receiving portion 29 to stand by. Therein, the washing solution is an ethanol aqueous solution of 70% concentration.

At the nucleic acid eluting process, the connecting pipette nozzle 36 is moved to the position of the first pipette tip on the tip rack 14c, and the pipette tip 15 is fit to the top end of the connecting pipette nozzle 36 by moving the nozzle 36 downward. The connecting pipette nozzle 36 connecting the pipette tip is moved to the position of the eluting solution bottle 20. The eluting solution bottle 20 contains purified water as the eluting solution. The eluting solution bottle 20 is preferably heated. An amount of the eluting solution capable of being used several times of eluting operation is sucked into the pipette tip 15 by sucking operation of the syringe pump 10. Successively, the pipette tip 15 is moved above the first treating container 24 on the container rack 23, and the eluting solution by an amount for one eluting operation is discharged from the pipette tip 15 into the first treating container 24 by delivering operation of the syringe pump 10. The pipette tip 15 containing the remaining amount of the eluting solution is moved to the liquid receiving portion 11 to stand by.

The nucleic acid trapping pipette tip standing by at the liquid receiving portion 28 is moved to the first treating container 24 on the container rack 23, and the eluting solution in the first treating container 24 containing the eluting solution is sucked into the nucleic acid trapping pipette tip 31. By doing so, the eluting solution is in contact with the solid phase substance to elute the nucleic acid adsorbed on the surface of the solid phase substance into the eluting solution. Operation that the eluting solution sucked in the pipette tip 31 is discharging into the original treating container 24 and then the discharged eluting solution is sucked into the same pipette tip 31 again is repeated predetermined cycles, and the nucleic acid trapping pipette tip 31 finally holding the eluting solution is moved to the position of the first purified product container 26 on the container rack 25. The eluting solution contained in the nucleic acid pipette tip 31 is discharged into the first purified product container 26 by delivering operation of the syringe pump 32. By doing so, the eluting solution containing the nucleic acid eluted from the solid phase substance is collected in the purified product container 26. The nucleic acid trapping pipette tip 31 after discharging the eluting solution is moved to the liquid receiving portion 28 to stand by.

Next, the pipette tip 15 in a state of holding the eluting solution is moved from the liquid receiving portion 11 to the first treating container 24, the eluting solution by an amount for the next eluting operation is discharged into the corresponding treating container. Successively, the nucleic acid trapping pipette tip 31 standing by at the liquid receiving portion 28 is moved to the first treating container 24, and the eluting solution in the treating container 24 is sucked into the nucleic acid trapping pipette tip 31 to perform the similar nucleic acid eluting operation as described above. After that the eluting solution containing the nucleic acid is collected in the first purified product container 26. The eluting solution supplying operation by the pipette tip 15 and the eluting operation by the nucleic acid trapping pipette tip 31 described above are repeated several cycles, for example, three cycles. The pipette tip 15 after completing of discharging the eluting solution is moved to the pipette tip detaching implement 27, and the used pipette tip 15 is detached from the connecting pipette nozzle 36. The connecting pipette nozzle 36 releasing the pipette tip is moved to the liquid receiving portion 11, and is sucked a very small amount of air in the top end of the nozzle after discharging water from the top end of the nozzle, and stands by at the position. On the other hand, the nucleic acid trapping pipette tip 31 after completion of several times of discharging the eluting solution containing the nucleic acid to the purified product container 26 is moved to the pipette tip detaching implement 27, and the used nucleic acid trapping pipette tip is detached from the movable nozzle 39. The movable nozzle 39 detaching the nucleic acid trapping pipette tip is moved to the liquid receiving portion 28, and is sucked a very small amount of air in the top end of the nozzle after discharging water from the top end of the nozzle, and stands by at the position.

Thus, the purifying operation of nucleic acid to the first sample is completed. After that, although the purifying apparatus 100 of FIG. 1 continues nucleic acid purifying operation to the second sample and the following samples, the operation is the same as in the example described above. However, although the first pipette tips on the respective pipette rack 14a, 14b, 14c, the first nucleic acid trapping pipette tip on the pipette rack 30, the first treating container 24 on the container rack 23 and the first purified product container on the container rack 25 are used for the first sample, the second items corresponding to these are used for the second sample and similarly the third items and the following items are used for the third sample and the following sample. Therefore, in the array of the purified product containers on the container rack 25, the purified and collected solutions containing nucleic acid are collected on a sample-by-sample basis according to the sample order. Since new items are used for every sample, contamination between the samples can be prevented.

Another example of the construction of the nucleic acid trapping pipette tip will be described below. The nucleic acid trapping pipette tip 31a of FIG. 8 has a plurality of silica blocks 45 as the solid phase substance in the inside. The silica block 45 is formed through sintering so as to have an outer diameter larger than the inner diameter of the top end of the pipette tip 31a. A disk-shaped preventive member 40c for preventing the solid phase substance from flowing out is press-fit into the head portion side of the nucleic acid pipette tip 31a. The material of this preventive member 40c is the same as that of FIG. 7.

A nucleic acid trapping pipette tip 31b of FIG. 9 has quartz wool 46 as the solid phase substance in the inside. A disk-shaped preventive member 40d is press-fit in the top-end side of the nucleic acid trapping pipette tip 31b, and a preventive member 40c is press-fit in the head portion side. These preventive members have many small through holes through which liquid and gas can pass through, and are made of poly(vinylidene fluoride).

Description will be made below on a test example in regard to the recovering ratio of nucleic acid in a case where commercial nucleic acid products are treated by the purifying apparatus of FIG. 1 using the various kinds of the nucleic acid trapping pipette tips shown in FIG. 7 to FIG. 9. The treated sample used was prepared by diluting pBR322DNA (a product of Fermentas Co.) to a predtermined concentration with a Tris-EDTA buffer. A predetermined amount of the washing solution was supplied to the treating container 24 twice in the washing process by the apparatus of FIG. 1, and a predetermined amount of the eluting solution (purified water) was supplied to the treating container 24 twice in the eluting process. Electrophoretic measurement was performed on collected solutions collected in different purified product containers 26 for respective samples to which different kinds of the nucleic acid trapping pipette tips were used. In the electrophortic measurement, agarose gel of 0.8% was used, and after dyeing with ethidium bromide intensities of bands were converted into numbers using a densitograph (a product of ATTO Co.). Recovering ratios were calculated based on amounts of nucleic acid before and after the treating operation.

According to the test results, the nucleic acid recovering ratios were 87% for the pipette tip of FIG. 7, 52% for the pipette tip of FIG. 8 and 80% for the pipette tip of FIG. 9. The treating time using the purifying apparatus of FIG. 1 was approximately 10 minutes per one sample.

A test example on the relationship between kind or property of nucleic acid and recovering ratio will be described below. Sample solutions were respectively prepared by diluting commercial products of λDNA (double-stranded linear DNA having 48502 basic length, a product of Fermentas Co.), pBR322DNA (double-stranded ring DNA having 4361 basic length, a product of Fermentas Co.) and MS2RNA (single-stranded linear RNA having 3569 basic length, a product of Bohringer Mannhiem Co.) to a predetermined concentration with a TE buffer. The samples were held to a sample rack 12, and the sample rack was set on the apparatus of FIG. 1. The DNAS were automatically recovered using the nucleic acid trapping pipette tips of FIG. 7 through the same process as in the above-mentioned test. Using part of the nucleic acid solution obtained in the purified product container 26 collected on a sample-by-sample basis, the electrophortic measurement was performed using agarose gel of 8%, and after dyeing with ethidium bromide intensities of bands were converted into numbers using a densitograph (a product of ATTO Co.). Recovering ratios were calculated based on amounts of nucleic acid before and after the treating operation. According to the test results, the nucleic acid recovering ratios were 68% for λDNA, 68 5 for pBR332 and 78% for MS2RNA. Although the recovering ratios are slightly different depending on the property of nucleic acid, it can be understood that nucleic acid can be recovered with ease and high recovering ratio by using the purifying apparatus of the present invention.

Description will be made below on a test for checking a DNA purifying condition in a case where blood serum coexists. A sample was prepared by adding the commercial product of λDNA (a product of Fermentas Co.) to human blood serum and further adding sodium dodecyl sulfate (1% in the final concentration) in order to perform the nuclease measures and bring close to the practical condition. The DNA was automatically recovered using the nucleic acid trapping pipette tips of FIG. 7 through the same process as in the above-mentioned test.

PCR treatment was performed to a recovered nucleic acid solution collected in each of the purified product containers 26. A PCR-purposed reagent kit, a product of Takara Shuzo Co., was used as a reagent at the PCR treatment, and amplification of 500 base specific to λDNA was tried by a λDNA control primer attendant on the kit. PCR was performed by repeating 25 cycles of denature at 94° C. for 30 seconds and annealing and polymerizing reaction at 68° C. for 30 seconds and further heating 68° C. for 7 minutes using TP3000 (a product of Takara Shuzo Co.) for the gene amplification system at PCR treating. After PCR treatment, the electrophortic measurement was performed using agarose gel of 1.5%, and dyeing with ethidium bromide was performed.

Comparison was performed by letting a case where the sample was directly PCR treated without purifying treatment using the purifying apparatus of FIG. 1 be Case A, a case where the sample was PCR treated after purifying treatment using the purifying apparatus of FIG. 1 be Case B, and a case where a sample was prepared so as to have a nucleic acid concentration equal to that of Case B without mixing the commercial product of λ DNA with blood serum and the sample was PCR treated be Case C. According to the test results, in Case A, the objective fragments of DNA could not be obtained due to obstruction of sodium dodecyl sulfate (SDS) and so on. On the other hand, in Case B, the objective fragments of DNA equivalent to in Case C could be obtained. It can be confirmed from the test results that the purifying apparatus of FIG. 1 can purify nucleic acid to the sample coexisting with blood serum.

Description will be made below on an test example for collecting plasmid DNA from cultured genetic recombined colon bacilli though purification. A pre-treatment is necessary for preparing a sample to be set in the purifying apparatus of FIG. 1. In the pre-treatment, an objective sample was obtained by culturing colon bacilli of E.coli HB101 recombined with pBR322DNA through genetic engineering means in a 1 ml LB culture culture medium at 37° C. for one night; gathering the bacilli by centrifugal separation; suspending the gathered bacilli in a 100 μ1 solution of 0.15 mol/l NaCl; adding a solution to the suspension and mixing it in order to break peptidoglycan in cell wall of the colon bacillus, the solution being prepared by adding lysozyme to 50 mmol/l glucose, 10 mmol/l EDTA, 25 m mol/l Tris-HCl (pH 8.0) so as to become 8 mg/ml; further adding 0.2 mol/l NaOH and a 1% SDS solution; leaving the solution for a predetermined time period; and then adding 5 mol/l potassium acetate to the solution.

The pre-treated sample was contained in a sample rack 12, and purifying operation of plasmid DNA was performed by the purifying apparatus of FIG. 1 using the nucleic acid trapping pipette tip of FIG. 7. Absorbance measurement was performed on the recovered solution collected to the purified product container 26 using a spectrophotometer. The result showed that a ratio of absorbance of 260 nm wavelength to absorbance of 280 nm wavelength was 1.96. Since absorption wavelength of nucleic acid is 260 nm and absorption wavelength of protein is 280 nm, it can be said that degree of purifying nucleic acid is high when the absorbance ratio is larger than 1.8. Further, the electrophortic measurement was performed using agarose gel of 0.8%, and dyeing with ethidium bromide was performed. Plasmide DNA and rRNA were observed in the purified solution from molecular weight markers carried at a time. Further, from digesting treatment of DNA by RNase and 260 nm absorbance of the plasmid DNA container after deposition treatment by an alcohol solution, it was found that the yield of plasmid DNA was 4.1 μg.

According to the present invention, since the purifying operation of nucleic acid can be automated and contacting time between a sample containing nucleic acid and the solid phase substance can be sufficiently secured, nucleic acid can be purified with a high yield. Further, according to the present invention, since the process of trapping nucleic acid to the solid phase substance, the process of washing the solid phase substance trapping the nucleic acid and the process of eluting the nucleic acid from the solid phase substance can be performed under the condition that the nucleic acid trapping pipette tip containing the solid phase substance is kept in connecting to the movable nozzle, purifying operation can be easily performed.

What is claimed is:

1. A method of purifying nucleic acid from a sample containing the nucleic acid, the method comprising the steps of:
detachably connecting a nucleic acid trapping pipette tip containing a solid phase substance containing silica to a liquid sucking-and-discharging movable nozzle;
sucking a mixture of a substance for enhancing binding of the nucleic acid to said solid phase substance into the nucleic acid trapping pipette tip connected to said liquid sucking-and-discharging movable nozzle from a predetermined container;
discharging the liquid in said nucleic acid trapping pipette tip after binding the nucleic acid in the sucked mixture to said solid phase substance;
washing said solid phase substance in the state of binding the nucleic acid and the inside of said nucleic acid trapping pipette tip by sucking a washing solution into said nucleic acid trapping pipette tip after discharging the liquid and then by discharging said washing solution from said nucleic acid trapping pipette tip;
sucking an eluting solution into said nucleic acid trapping pipette tip after washing; and
discharging the eluting solution containing the nucleic acid removed from said solid phase substance into a purified product container.

2. A method of purifying nucleic acid according to claim 1, wherein said nucleic acid trapping pipette tip connected to said liquid sucking-and-discharging movable nozzle is exchanged to a new nucleic acid trapping pipette tip every change of a sample to be treated.

3. A method of purifying nucleic acid according to claim 1, wherein the contact between said mixture and said solid phase substance is repeated plural cycles by once discharging said mixture to said predetermined container after sucking said mixture into said nucleic acid trapping pipette tip and then by sucking said mixture into the same nucleic acid trapping pipette tip again.

4. A method of purifying nucleic acid according to claim 1, wherein in the step of washing said solid phase substance and the inside of said nucleic acid trapping pipette tip, the first washing solution is discharged from said nucleic acid trapping pipette tip and then a new washing solution is sucked into and discharged from said nucleic acid trapping pipette tip.

5. A method of purifying nucleic acid according to claim 1, the method further comprising the steps of detachably connecting a liquid pipetting tip to a liquid transferring nozzle; pipetting a washing solution from a washing solution bottle into said predetermined container using said liquid pipetting tip; and sucking the washing solution pipetted in said predetermined container into said nucleic acid trapping pipette tip.

6. A method of purifying nucleic acid according to claim 1, the method further comprising the steps of detachably connecting a liquid pipetting tip to a liquid transferring nozzle; pipetting an eluting solution from an eluting solution bottle into said predetermined container using said liquid pipetting tip; and sucking the eluting solution pipetted in said predetermined container into said nucleic acid trapping pipette tip.

7. A method of purifying nucleic acid according to claim 6, wherein said liquid pipette tip pipettes the eluting solution by dividing into plural times into said predetermined container, and said nucleic acid trapping pipette tip sucks the eluting solution from said predetermined container plural times.

8. A method of purifying nucleic acid according to claim 1, wherein said binding enhancer is guanidine hydrochloride.

9. A method of purifying nucleic acid according to claim 1, wherein said washing solution is ethyl alcohol aqueous solution.

10. An apparatus for purifying nucleic acid from a sample containing the nucleic acid, which comprises:
a nucleic acid trapping pipette tip containing a solid phase substance containing silica capable of contacting with a liquid;
a liquid sucking-and-discharging movable nozzle detachably connecting said nucleic acid trapping pipette tip;
a treating container capable of containing a mixture of a substance for enhancing binding of said nucleic acid with said solid phase substance and a sample containing the nucleic acid;
means for supplying a washing solution to said treating container;
means for supplying a eluting solution to said treating container;
a purified product container for receiving a purified product of the nucleic acid;
transferring means for connecting said nucleic acid trapping pipette tip in an unused state to said liquid sucking-and-discharging movable nozzle and for moving said nucleic acid trapping pipette tip in a connected state to positions of said treating container and said purified product container;
liquid sucking-and-discharging operating means for sucking and discharging said mixture from and to said nucleic acid trapping pipette tip connected to said liquid sucking-and-discharging movable nozzle, and sucking and discharging said washing solution, and then sucking and discharging said eluting solution; and
tip detaching means for detaching said nucleic acid trapping pipette tip from said liquid sucking-and-discharging movable nozzle after discharging the eluting solution from said nucleic acid trapping pipette tip to said purified product container.

11. An apparatus for purifying nucleic acid according to claim 10, wherein said nucleic acid trapping pipette tip comprises a liquid communicable preventive member for preventing said solid phase substance in said nucleic acid trapping pipette tip from flowing out.

12. An apparatus for purifying nucleic acid according to claim 11, wherein said preventive member is made of poly(vinylidene fluoride).

13. An apparatus for purifying nucleic acid according to claim 11, wherein said preventive member is arranged at a position nearer to a connecting terminal with said liquid sucking-and-discharging movable nozzle than a containing region of said solid phase substance, and has an insertion assisting guide formed therein.

14. An apparatus for purifying nucleic acid according to claim 10, which comprises a liquid pipette tip for pipetting said sample and said binding enhancer to said treating container.
